(19) United States
(12) Patent Application Publication
(10) Pub. No.: US 2013/0336452 A1
(43) Pub. Date: Dec. 19, 2013

(54) RADIATION IMAGING APPARATUS, RADIATION IMAGING SYSTEM, RADIATION IMAGING METHOD, AND PROGRAM

(71) Applicant: CANON KABUSHIKI KAISHA, Tokyo (JP)

(72) Inventors: Daizo Matsuda, Funabashi-shi (JP); Keiji Tsuchiya, Kawasaki-shi (JP)

(21) Appl. No.: 13/916,742

(22) Filed: Jun. 13, 2013

(30) Foreign Application Priority Data
Jun. 13, 2012 (JP) 2012-133905
Jun. 3, 2013 (JP) 2013-117392

(51) Int. Cl. H05G 1/62

(52) U.S. Cl. CPC H05G 1/62
USPC 378/62

(57) ABSTRACT

A preliminary dark image acquisition unit 43 images a first preliminary dark image in each accumulation time, in advance, by accumulating a charge in at least one or more different accumulation times in a sensor 4 under a condition that the X ray is not irradiated. An X-ray image imaging unit 41 controls an irradiation of an X ray to image an X-ray image, selects an accumulation time corresponding to an X-ray irradiation time from among the accumulation times, and sets an accumulation time of a charge in the sensor 4 when the X-ray image is imaged to the selected accumulation time. The X-ray image imaging unit 41 images the X-ray image by accumulating the charge in the sensor 4 for the selected accumulation time. An offset correction unit 46 performs offset correction on the X-ray image based on the first preliminary dark image.

### BACKGROUND OF THE INVENTION

1. Field of the Invention

The present invention relates to a device, a system, a method, and a program that image a radiation image.

2. Description of the Related Art

At present, regarding X-ray still image imaging systems of the medical field, a film type of irradiating a patient as a subject with the X rays and exposing an X-ray image passing through the patient on a film has been mainly used. Since a film has a function of displaying and recording information, has a large area, has a high gradation property, and is easily treated due to lightweight, the film has been spread worldwide. In contrast, the film has problems of troublesomeness of a development process to be necessarily performed, a long-term storage place, manpower and time necessary for search, and the like.

In recent years, a demand for digitizing the X-ray still image imaging systems has increased in hospitals. X-ray image imaging apparatuses (FPD: Flat Panel Detector) have been spread which convert an X-ray dosage into an electric signal using an X-ray detection apparatus in which solid-state imaging elements are arrayed in a two-dimensional matrix form, instead of a film. Since the X-ray image imaging apparatus can substitutes an X-ray image with digital information, the X-ray image substituted with the digital information (hereinafter, referred to as an X-ray image) can be transmitted instantaneously to a distant place. By transmitting the X-ray image, it is possible to obtain the advantage of receiving an advanced diagnosis equivalent to that of a downtown university hospital even when a patient is located at a distant place. Further, by not using a film, it is possible to obtain the advantage of reducing a storage space of films in a hospital. In the future, by introducing excellent image processing technologies, a possibility of an automatic diagnosis using a computer without a medical doctor is expected.

Such an X-ray image imaging apparatus includes a photoelectric conversion circuit in which a plurality of photoelectric conversion elements converting radial rays into electric signals are arrayed in a matrix form, and a reading circuit that reads the electric signals obtained through the conversion from the photoelectric conversion circuit. When a subject is irradiated with X rays, photoelectric conversion relevant to the X rays passing through the subject is performed by each photoelectric conversion element in the photoelectric conversion circuit and a charge corresponding to the X-ray dosage is accumulated in each photoelectric conversion element. By driving each signal line of the photoelectric conversion circuit and appropriately controlling switching elements connected to the photoelectric conversion elements, the charge accumulated in each photoelectric conversion element is sequentially read as an electric signal by the reading circuit, the electric signal is amplified, and the amplified electric signal is output.

By performing the operation in the above-described way, the X-ray image can be read. However, an offset produced in the photoelectric conversion circuit or the reading circuit is contained in the X-ray image itself. There are several causes of the offset. The causes include (A) a dark current of the photoelectric conversion element, (B) a leakage current of the switching element, (C) an offset voltage of an amplifier of the reading circuit and the like. These causes vary due to a temperature variation and a difference in an accumulation time.

As described above, the offset is contained in the X-ray image. Therefore, the offset component has to be eliminated. A process of eliminating the offset component is called offset correction. In the past, the offset correction has been performed by imaging a dark image without irradiation of a subject with the X rays after irradiating the subject with the X rays and imaging the X-ray image and by subtracting the dark image from the X-ray image.

In the above-described offset correction, however, the dark image is acquired after the irradiation of the X rays. Therefore, there is a problem that a time necessary to display an image from the irradiation of the X rays is prolonged. In order to resolve this problem, Japanese Patent No. 3,190,328 discloses a method of acquiring a dark image at the time of state transition of an X-ray image imaging apparatus before radiation of the X rays. In the method disclosed in Japanese Patent No. 3,190,328, a dark image continues to be acquired periodically, and thus the dark image used for the actual correction is the dark image acquired at the timing close to the imaging.

However, a time at which the irradiation of the X-ray is performed is unclear even in the method of continuously acquiring the dark image periodically. Accordingly, when the radiation of the X rays is required during the acquisition of the dark image, the radiation of the X rays is not performed until the acquisition of the dark image completes. Thus, there is a problem that an immediacy of the requirement for the radiation of the X rays is poor. In order to resolve this problem, Japanese Patent No. 4,104,443 discloses a method of acquiring a dark image, only when a specific request is made, and using the dark image acquired in response to the specific request for the offset correction.

However, a property of the offset of the X-ray image imaging apparatus varies due to a temperature variation and a difference in an accumulation time. Accordingly, an occurrence state of the dark current varies because a condition at the time when actual imaging of the X-ray image is performed is different from a condition at the time of acquisition of the dark image used to perform the offset correction. Accordingly, in the technique disclosed in Japanese Patent No. 4,104,443, an error caused due to the temperature variation and the difference in the accumulation time of the X-ray image may not be corrected.

Accordingly, the present invention provides a radiation imaging apparatus capable of reliably performing offset correction.

### SUMMARY OF THE INVENTION

According to an aspect of the invention, an radiation imaging apparatus includes: a first preliminary dark image imaging unit that images a first preliminary dark image in each accumulation time, in advance, by accumulating a charge in one or more different accumulation times by a detection unit that detects a radial ray and accumulates the charge, under a condition that the radial ray is not irradiated by an irradiation unit; an irradiation control unit that controls the irradiation unit to irradiate the radial ray to image a radiation image; a selection unit that selects an accumulation time corresponding to an irradiation time of the radial ray by the irradiation unit from among the accumulation times; a setting unit that sets an accumulation time of a charge in the detection unit when the radiation image is imaged to the accumulation time selected by the selection unit; a radiation image imaging unit that images the radiation image by accumulating, by the detection unit, the charge in the accumulation time set by the setting unit; and an offset correction unit that performs offset correction on the radiation image based on the first preliminary dark image.

Accordingly, the invention can reliably perform the offset correction.

Further features of the present invention will become apparent from the following description of exemplary embodiments with reference to the attached drawings.

### BRIEF DESCRIPTION OF THE DRAWINGS

FIG. 1A illustrates the configuration of an X-ray image imaging system according to an embodiment of the invention.

FIG. 1B illustrates the configuration of an X-ray image imaging system according to a modification example of the embodiment of the invention.

FIGS. 2A and 2B are timing charts illustrating an operation of the X-ray image imaging system according to a first embodiment of the invention.

FIGS. 3A and 3B are flowcharts illustrating a process of the X-ray image imaging system according to the first embodiment of the invention.

FIG. 4 is a flowchart illustrating a process of the X-ray image imaging apparatus according to the second embodiment of the invention.

FIG. 5 is a timing chart illustrating an example of timings of accumulation of a charge and reading of the charge in a sensor according to a second embodiment of the invention.

### DESCRIPTION OF THE EMBODIMENTS

Preferred embodiments of the present invention will now be described in detail in accordance with the accompanying drawings. In the embodiments to be described below, an example of an X-ray image imaging apparatus which uses X rays will be described, but the invention is not limited thereto. For example, the invention is not limited to the X rays, but radial rays such as α rays, β rays, γ rays and luminous rays are included in the scope of the invention. Apparatuses processing an image imaged using such radial rays are also included in the scope of the invention.

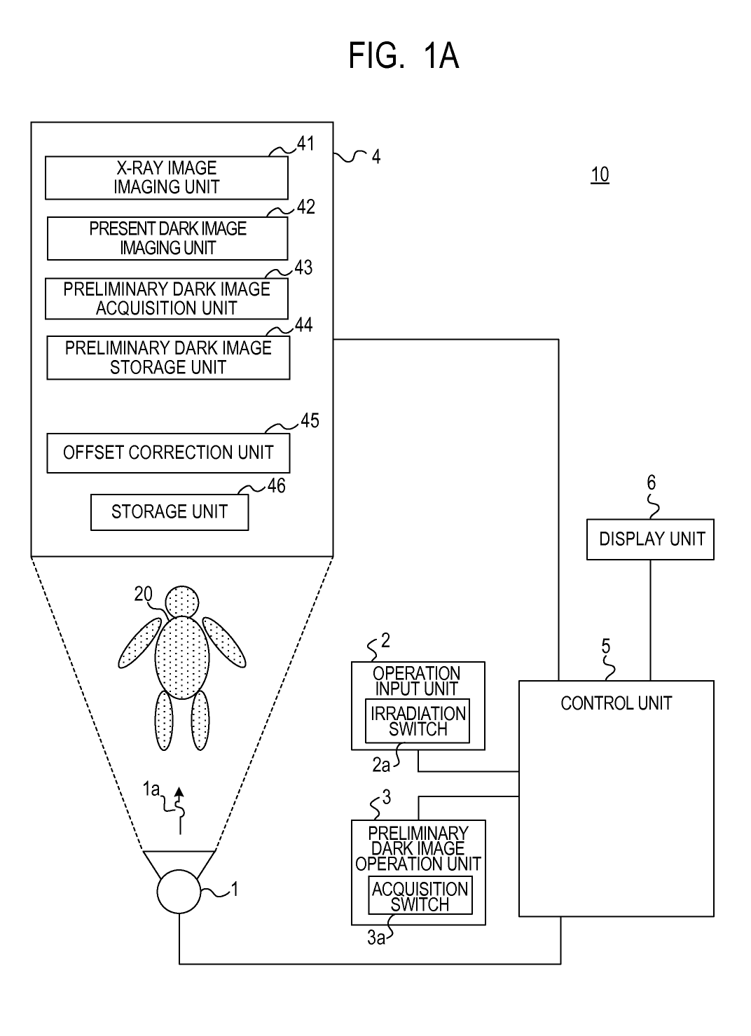

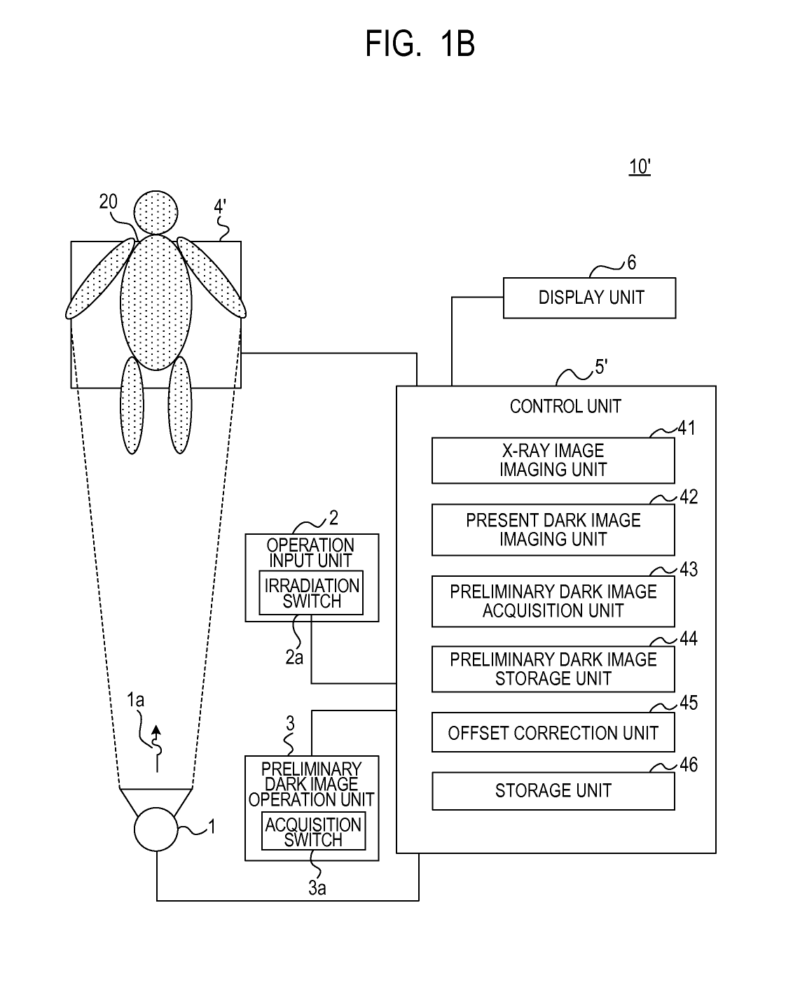

First, a first embodiment of the invention will be described. FIG. 1A illustrates the configuration of an X-ray image imaging system according to the first embodiment of the invention. FIG. 1B illustrates the configuration of an X-ray image imaging system according to a modification example of the embodiment of the invention. The X-ray image imaging system 10 according to the first embodiment of the invention will be described with reference to FIG. 1A. As illustrated in FIG. 1A, an X-ray image imaging system 10 according to the first embodiment includes an X-ray generation unit 1, an operation input unit 2, a preliminary dark image operation unit 3, a sensor 4 (X-ray image imaging apparatus), a control unit 5, and a display unit 6. The sensor 4 includes an X-ray image imaging unit 41, a present dark image imaging unit 42, a preliminary dark image acquisition unit 43, a preliminary dark image storage unit 44, an offset correction unit 45, and a storage unit 46. The X-ray image imaging system 10 is a configuration example of a radiation imaging system.

The X-ray generation unit 1 is a unit that generates an X ray 1a for a subject (examinee) 20 and includes, for example, an X-ray tube. The operation input unit 2 is used when the user operate the X-ray image imaging system 10 to radiate an X ray from the X-ray generation unit 1. The operation input unit 2 includes a setting unit that sets imaging conditions (a tube voltage, a tube current, a radiation time, and the like) and an irradiation switch 2a. For example, when the tube voltage, the tube current, the irradiation time, and the like are set according to an imaging part of the subject (examinee) 20, and then it is detected that the irradiation switch 2a is turned on, the control unit 5 causes the X-ray generation unit 1 to radiate the X ray corresponding to the imaging conditions. In view of this, the control unit 5 functions as an irradiation control unit for the radial ray. The preliminary dark image operation unit 3 is used when the user preliminarily acquires a dark image. The preliminary dark image operation unit 3 includes a setting unit that sets imaging conditions used to image a preliminary dark image and an acquisition switch 3a.

The sensor 4 performs imaging of an X-ray image of the X ray transmitted through the subject 20 at the time of the irradiation of the X ray 1a from the X-ray generation unit 1, imaging of a dark image in a state in which the X ray 1a is not irradiated from the X-ray generation unit 1, that is, a dark state, and offset correction to generate a corrected X-ray image.

In the sensor 4, for example, pixels including a photoelectric conversion element and a TFT are arrayed two-dimensionally and, for example, a phosphor is installed on each pixel. The X ray incident on the sensor 4 is converted into the visible light by the phosphor, the converted visible light is incident on the photoelectric conversion element of each pixel, and a charge according to the visible light is generated in each photoelectric conversion element. In this embodiment, a conversion element that converts the incident X ray into a charge is formed by the phosphor and the photoelectric conversion element described above. However, a so-called direct conversion type conversion element that directly converts the incident X ray into a charge without providing the phosphor may be configured. In the sensor 4 according to this embodiment, the conversion elements are arrayed two-dimensionally, so that the sensor 4 can image an X-ray image and a present dark image by accumulation of a charge in each conversion element and reading of the charge. The sensor 4 has a function of preliminarily imaging one or more preliminary dark images and a function of storing the imaged preliminary dark image, an average pixel value and the like of the preliminary dark image.

The storage unit 46 includes a volatile memory such as a RAM. The storage unit 46 stores images imaged by the X-ray image imaging unit 41 and the present dark image imaging unit 42 and an average pixel value thereof. Further, the sensor 4 includes a memory (not shown) and the memory includes a non-volatile memory such as a flash ROM. A program or the like controlling the entire sensor 4 is written on the memory. Based on the program, the imaging of the X-ray image, the imaging of a present dark image, the offset correction, the imaging of the preliminary dark image, and the like are performed by the sensor 4.

The control unit 5 controls the X-ray generation unit 1 and the sensor 4 based on an operation of the operation input unit 2, receives the X-ray image generated and subjected to the offset correction by the sensor 4, performs image processing or the like on the received X-ray image, and then displays the processed X-ray image on the display unit 6. The display unit 6 also displays an operation UI or the like.

The X-ray image imaging unit 41 images an X-ray image by accumulating charges in the photoelectric conversion elements during the irradiation of the X ray and reading the charges from the photoelectric conversion elements after the irradiation of the X ray, and stores the imaged X-ray image in the storage unit 46.

The present dark image imaging unit 42 performs a process after the X-ray image imaging unit 41 images the X-ray image. That is, the present dark image imaging unit 42 images a dark image by accumulating charges in the photoelectric conversion elements when the X ray is not irradiated and then reading the charges from the photoelectric conversion elements. Here, the dark image imaged after the imaging of the X-ray image is referred to as a present dark image. The present dark image imaging unit 42 calculates an average pixel value of a predetermined region of the imaged present dark image and stores the calculated average pixel value of the present dark image in the storage unit 46. An accumulation time at the time of the imaging of the present dark image is set to be shorter than that at the time of the imaging of the X-ray image. A reading time of the present dark image is set to be shorter than that at the time of the imaging of the X-ray image.

The preliminary dark image acquisition unit 43 images a first dark image in a predetermined first accumulation time and images a second dark image in a predetermined second accumulation time after the imaging the first dark image. Here, the dark image imaged at the first accumulation time is referred to as a first preliminary dark image and a dark image imaged at the second accumulation time is referred to as a second preliminary dark image. The preliminary dark image acquisition unit 43 calculates an average pixel value of a predetermined region of the second preliminary dark image. The preliminary dark image acquisition unit 43 images the first and second preliminary dark images once or more.

The preliminary dark image storage unit 44 includes a non-volatile memory such as a flash ROM and stores, as a set, an accumulation time for reading of the first preliminary dark image, the first preliminary dark image, an accumulation time for reading of the second preliminary dark image, and the average pixel value of the predetermined region of the second preliminary dark image. The preliminary dark image storage unit 44 stores n sets. However, since the accumulation time at the time of reading of the second preliminary dark image is normally same, this accumulation time may be excluded from the set and may be stored separately as one independent value.

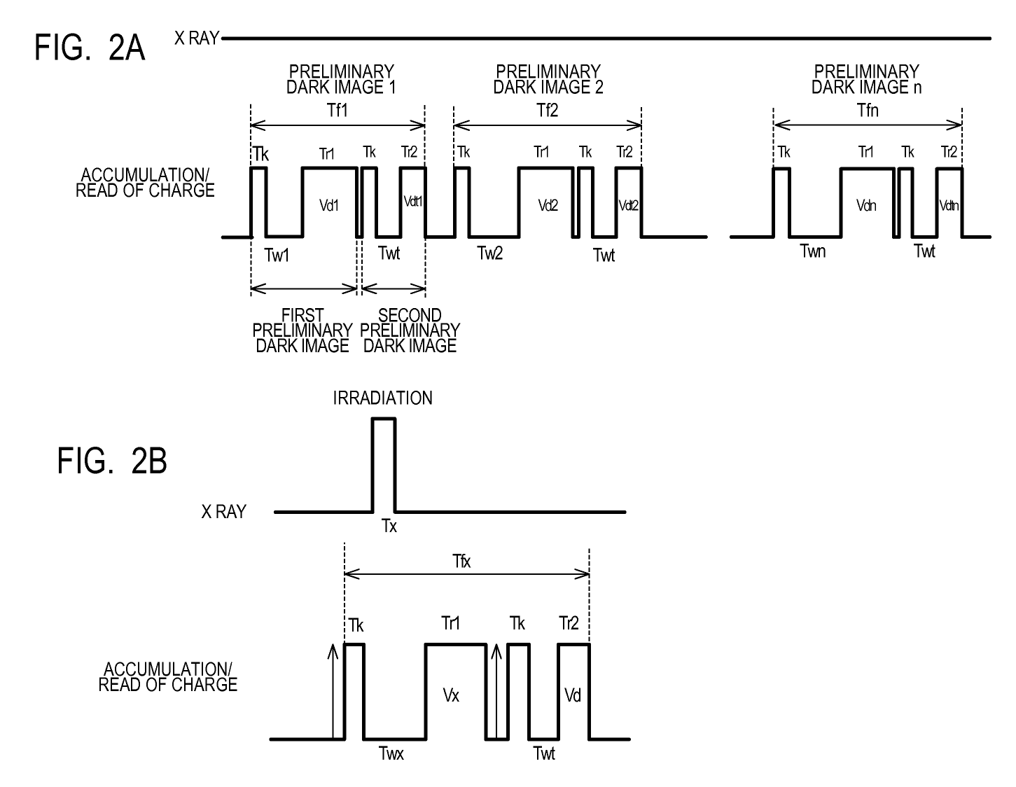

Next, a process of imaging the first preliminary dark image and the second preliminary dark image by the preliminary dark image acquisition unit 43 will be described with reference to FIG. 2A. FIG. 2A is a timing chart illustrating timings of the accumulation of the charge and the reading of the charge in each conversion element of the sensor 4. As illustrated in FIG. 2A, the preliminary dark image acquisition unit 43 images the first and second preliminary dark images by performing accumulation and reading of the charge in each conversion element of the sensor 4. FIG. 2A illustrates an example in which the first and second preliminary dark images are imaged n times and an accumulation time at the time of the imaging of the first preliminary dark image is changed at each imaging. Vd1 denotes the first preliminary dark image of a first time, Vdt1 denotes the second preliminary dark image of the first time, Vd2 denotes the first preliminary dark image of a second time, Vdt2 denotes the second preliminary dark image of the second time, Vdn denotes the first preliminary dark image of an n-th time, and Vdtn denotes the second preliminary dark image of the n-th time. Here, n is any integer equal to or greater than 1.

In FIG. 2A, before accumulation is performed to image the first and second preliminary dark images, it is necessary to perform a process of reading unnecessary charges occurring due to a dark current. Tk is assumed to be a reading time of the unnecessary charges. Twn is assumed to be an accumulation time of the first preliminary dark image of the n-th time and Tr1 is assumed to be a first preliminary dark image reading time. The accumulation time at the time of the reading of the second preliminary dark image is normally the same. On the assumption that Twt is an accumulation time when the second preliminary dark image is read and Tr2 is a reading time of the second preliminary dark image, an imaging time Tfn of the first and second preliminary dark images of the n-th time is expressed by Equation 1 below:

Tfn=2Tk+Twn+Tr1+Twt+Tr2 Equation 1

The accumulation time TWn of the first preliminary dark image of the n-th time and the accumulation time Twt of the second preliminary dark image are set such that Equation 2 below is normally satisfied:

Twt<Twn Equation 2

The reading time Tr1 of the first preliminary dark image and the reading time Tr2 of the second preliminary dark image are set such that Equation 3 below is satisfied:

Tr2≦Tr1 Equation 3

Here, for example, the reading time Tk is 200 ms. The accumulation time Tw1 at the time of the reading of the first preliminary dark image of the first time is 200 ms. The accumulation time Tw2 at the time of the reading of the first preliminary dark image of the second time is 400 ms. The accumulation time Tw3 at the time of the reading of the first preliminary dark image of the third time is 1000 ms. The reading time Tr1 of the first preliminary dark image is 400 ms. The accumulation time Twt at the time of the reading of the second preliminary dark image is 1 ms. The reading time Tr2 of the second preliminary dark image is 200 ms.

The X-ray image imaging unit 41 receives information on the irradiation time of the imaging conditions set in the operation input unit 2 via the control unit 5 and determines an accumulation time at the time of the imaging of the X-ray image based on the received information on the irradiation time. Specifically, when the X-ray image imaging unit 41 receives the information on the irradiation time, the X-ray image imaging unit 41 selects the accumulation time which is equal to or greater than the X-ray irradiation time and is the closest to the X-ray irradiation time from among the accumulation times at the time of the imaging of the first preliminary dark images, stored in the preliminary dark image storage unit 44. For example, if the accumulation times at the time of the imaging of the first preliminary dark images are 200 ms, 400 ms, and 1000 ms, the X-ray image imaging unit 41 selects 400 ms as accumulation time when the irradiation time is 300 ms, and the X-ray image imaging unit 41 selects 1000 ms as the accumulation time when the irradiation time is 500 ms. In view of this, the X-ray image imaging unit 41 functions as a selection unit for an accumulation time. The X-ray image imaging unit 41 sets the same time as the accumulation time of the selected first preliminary dark image as the accumulation time at the time of the imaging of the X-ray image and images an X-ray image. In view of this, the X-ray image imaging unit 41 functions as a setting unit for an accumulation time. The X-ray image imaging unit 41 stores the imaged X-ray image in the storage unit 46.

The present dark image imaging unit 42 sets the same time as the accumulation time Twt, stored in the preliminary dark image storage unit 44, at the time of the imaging of the second preliminary dark image as the accumulation time at the time of the imaging of the present dark image, and then images a present dark image. After the present dark image is imaged, an average pixel value of a predetermined region of the preset dark image is calculated. The present dark image imaging unit 42 stores the calculated average pixel value in the storage unit 46.

Next, the operations of the X-ray image imaging unit 41 and the present dark image imaging unit 42 will be described with reference to FIG. 2B. FIG. 2B is a timing chart illustrating an irradiation timing of the X ray and timings of the accumulation of the charge and the reading of the charge in each conversion element of the sensor 4.

In the example illustrated in FIG. 2B, the accumulation of the charge and the reading of the charge in each conversion element of the sensor 4 are performed so that the X-ray image imaging unit 41 images the X-ray image and the present dark image imaging unit 42 images the present dark image. The timing chart of FIG. 2B illustrates a case in which the X-ray image is first imaged and the present dark image is subsequently imaged.

In FIG. 2B, before accumulation is performed to image an X-ray image and a present dark image, it is necessary to perform a process of reading unnecessary charges occurring due to a dark current, as in FIG. 2A. Tk is assumed to be a reading time for the unnecessary charges. Tx is assumed to be an X-ray irradiation time, Twx is assumed to be an accumulation time of an X-ray image, and Tr1 is assumed to be a reading time of the X-ray image. Twt is assumed to be an accumulation time when the present dark image imaged after the reading of the X-ray image, and Tr2 is assumed to be a reading time of the present dark image. Here, X-ray irradiation Tx is an irradiation time set in the operation input unit 2, and the accumulation time Twx at the time of the reading of the X-ray image is assumed to be an accumulation time when the first preliminary dark image selected by the X-ray image imaging unit 41 is imaged. The accumulation time at the time of the reading of the present dark image is the same as the accumulation time at the time of the reading the second preliminary dark image. An entire imaging time Tfx of the X-ray image and the present dark image is expressed by Equation 4 below:

Tfx=2Tk+Twx+Tr1+Twt+Tr2 Equation 4

The X-ray irradiation time Tx and the accumulation time Twx at the time of the imaging of the X-ray image are set such that a relation expressed by Equation 5 below is satisfied:

Tx<Twx Equation 5

The X-ray image imaging unit 41 selects the accumulation time Twn which is equal to or greater than the X-ray irradiation time Tx and is the closest to the X-ray irradiation time Tx from among the accumulation times of the first dark image, stored in the preliminary dark image storage unit 44. Then, the X-ray image imaging unit 41 sets the accumulation time Twx for imaging the X-ray image to be the same as the selected accumulation time Twn. Accordingly, Equation 4 becomes Equation 6 below:

Tfx=2Tk+Twn+Tr1+Twt+Tr2 Equation 6

Here, for example, when the X-ray irradiation time Tx is 300 ms, the accumulation time of the first dark image which is equal to or greater than the X-ray irradiation time of 300 ms and is the closest to the X-ray irradiation time of 300 ms is 400 ms. On the assumption that the accumulation time Twt of the present dark image is set to 1 ms and the reading time Tr2 of the present dark image is set to 200 ms, the entire imaging time Tfx of the X-ray image and the present dark image becomes 1401 ms.

In the X-ray image imaging method according to the related art, however, the accumulation time Twx of the X-ray image and the accumulation time Twt of the present dark image are equally 400 ms, and the reading time Tr1 of the X-ray image and the reading time Tr2 of the present dark image are equally 400 ms. Therefore, the entire imaging time Tfx of the X-ray image and the dark image become 2000 ms. Accordingly, in the X-ray image imaging method according to this embodiment, the entire imaging time Tfx can be shorter by about 600 ms, as compared with the X-ray image imaging method according to the related art.

In this embodiment, as expressed in Equation 2, the accumulation time Twt of the present dark image is set to be shorter than the accumulation time Twx of the X-ray image. Therefore, the imaging time Tfx can be shortened. Accordingly, a time from the irradiation of the X ray to the display of the image can be shortened.

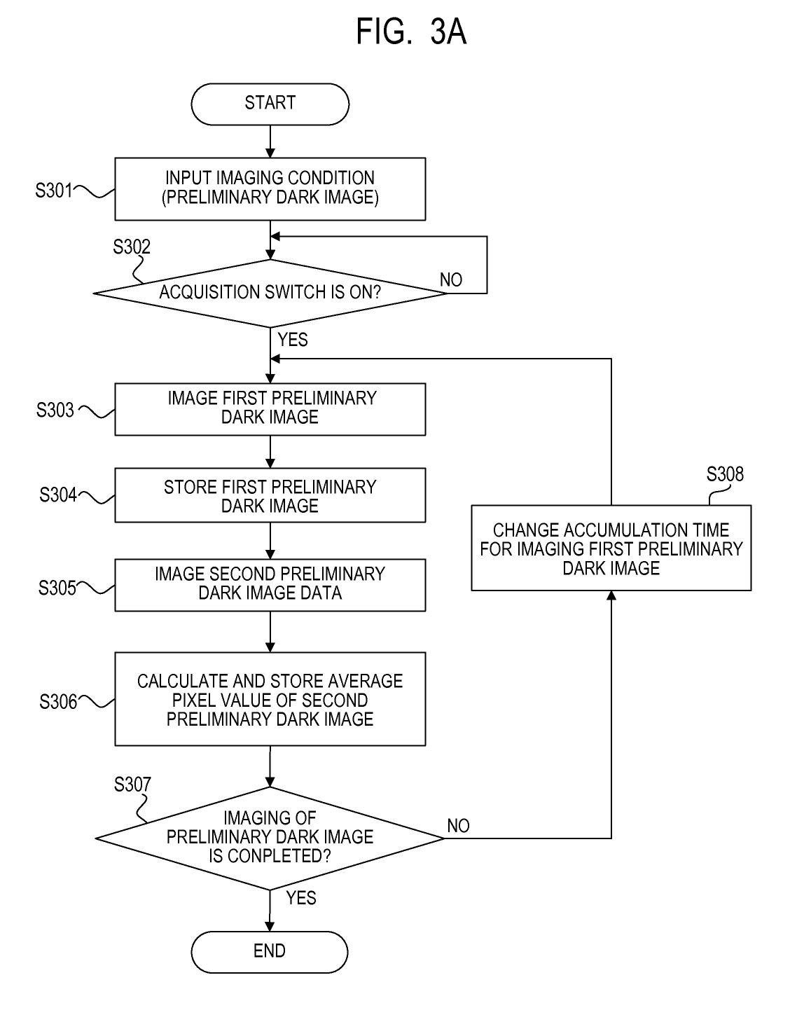

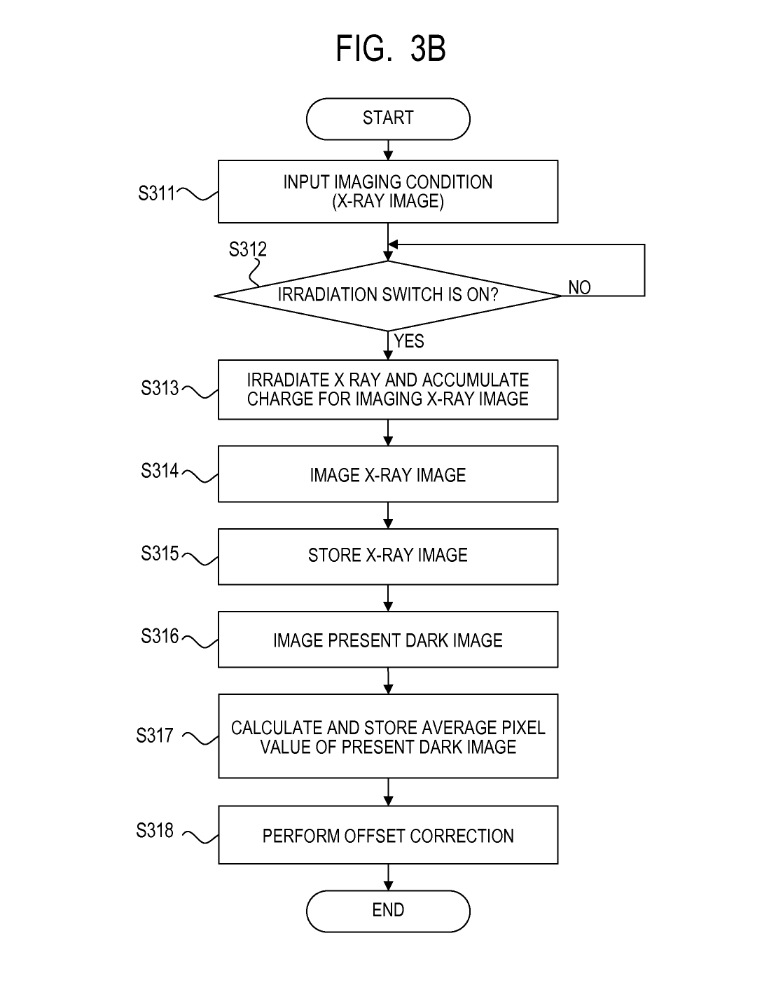

The offset correction unit 46 performs offset correction on the X-ray image based on the first preliminary dark image stored in the preliminary dark image storage unit 44, the average pixel value of the second preliminary dark image stored in the preliminary dark image storage unit 44, the X-ray image stored in the storage unit 46 and the average pixel value of the present dark image stored in the storage unit 46. The offset correction will be described in detail with reference to FIG. 3B. When the offset correction unit 46 performs the offset correction, the sensor 4 transmits the image subjected to the offset correction to the control unit 5 via a communication circuit (not shown). The control unit 5 performs image processing or the like on the image subjected to the offset correction and causes the display unit 6 to display the image.

The memory (not shown) stores a program or the like necessary for processes to be described below in FIGS. 3A and 3B and the other controls of the X-ray image imaging system 10.

Next, the process of the X-ray image imaging system 10 according to the first embodiment will be described with reference to FIGS. 3A and 3B. FIG. 3A is a flowchart illustrating a process of imaging the first preliminary dark image and the second preliminary dark image. The process illustrated in FIG. 3A is, for example, a process that is performed at a timing before factory shipment of the X-ray image imaging system 10.

In step S301, imaging conditions (accumulation time) are input into the preliminary dark image acquisition unit 43 in response to a user's operation of the preliminary dark image operation unit 3 and stored in the preliminary dark image storage unit 44 via the control unit 5. Here, for example, the accumulation times (200 ms, 400 ms, and 1000 ms) at the time of the imaging of the first preliminary dark image and the accumulation time (1 ms) at the time of the imaging of the second preliminary dark image are input as the imaging conditions and are stored in the preliminary dark image storage unit 44.

In step S302, the preliminary dark image acquisition unit 43 determines whether the user operates the acquisition switch 3a to turn on the acquisition switch 3a. When the acquisition switch 3a is turned on, the process proceeds to step S303. Conversely, when the acquisition switch 3a is in an off-state, the process returns to step S302 and stands by until the acquisition switch 3a is turned on.

In step S303, the preliminary dark image acquisition unit 43 images the first preliminary dark image in the accumulation time of 200 ms stored in step S301, after reading unnecessary charges occurring due to a dark current. In step S304, the preliminary dark image acquisition unit 43 stores the imaged first preliminary dark image in the preliminary dark image storage unit 44. Step S303 is an example of a process of imaging a first preliminary dark image in FIG. 2A.

In step S305, the preliminary dark image acquisition unit 43 images the second preliminary dark image in the accumulation time of 1 ms stored in step S301, after reading unnecessary charges occurring due to a dark current. In step S306, the preliminary dark image acquisition unit 43 calculates the average pixel value of the predetermined region of the imaged second preliminary dark image and stores the average pixel value of the second preliminary dark image for each accumulation time of the first preliminary dark image in the preliminary dark image storage unit 44. Step S305 is an example of a process of imaging a second preliminary dark image in FIG. 2A.

In step S307, the preliminary dark image acquisition unit 43 determines whether the imaging of the first preliminary dark image and the second preliminary dark image with respect to all of the accumulation times (200 ms, 400 ms, and 1000 ms) stored in the preliminary dark image storage unit 44 in step S301 is completed. When the imaging of the preliminary dark images with respect to all of the accumulation times is not completed, the accumulation time at the time of the imaging of the first preliminary dark image is changed in step S308 and the process returns to step S303. Conversely, when the imaging of the first preliminary dark image with respect to all of the accumulation times is completed, the preliminary dark image acquisition unit 43 finishes the process of imaging the first preliminary dark image and the second preliminary dark image.

When the processes proceeds from step S307 to step S308, in step S308, the preliminary dark image acquisition unit 43 changes the accumulation time for imaging the first preliminary dark image to an accumulation time in which the imaging is not performed among the accumulation times stored in the preliminary dark image storage unit 44 in step S301 and images the first preliminary dark image in step S303. That is, in this embodiment, by imaging the first preliminary dark image in the order of the accumulation times, 200 ms, 400 ms, and 1000 ms, the process of imaging the first preliminary dark image and the second preliminary dark image ends.

In this embodiment, as illustrated in FIG. 3A, the case has been described in which the first preliminary dark image and the second preliminary dark image are imaged in step S301 to step S308 before the imaging of the X-ray image, for example, before the factory shipment of the X-ray image imaging system 10, but the invention is not limited thereto. The first preliminary dark image and the second preliminary dark image may be imaged, for example, at the time of factory shipment of the X-ray image imaging system 10, at the time of installation of the X-ray image imaging system 10, or at an interval between the imaging of the X-ray image. The preliminary dark image storage unit includes a non-volatile memory such as a flash ROM. However, the invention is not limited to the non-volatile memory as long as the preliminary dark images are imaged during the imaging of the X-ray image but may include a volatile memory such as a RAM.

FIG. 3B is a flowchart illustrating a process of imaging the X-ray image, a process of imaging the present dark image and a subsequent offset correction process based on the first preliminary dark image and the average pixel value of the second preliminary dark image. A process illustrated in FIG. 3B is a process that is performed when a patient is imaged.

In step S311, the X-ray image imaging unit 41 receives information on the irradiation time of the imaging conditions set in the operation input unit 2 via the control unit 5 and determines the accumulation time at the time of the X-ray image based on the received information on the irradiation time. The method of determining the accumulation time has been described above with respect to the X-ray image imaging unit 41. Here, Twx in FIG. 2B is set as the accumulation time. The present dark image imaging unit 42 sets the accumulation time for imaging the present dark image to the accumulation time Twt (1 ms) for imaging the second preliminary dark image, stored in the preliminary dark image storage unit 44. The X-ray irradiation time Tx is set in the X-ray generation unit via the control unit 5.

In step S312, the control unit 5 determines whether the irradiation switch 2a is operated by the user and the irradiation switch 2a is thus turned on. When the irradiation switch 2a is turned on, the process proceeds to step S313. On the other hand, when the acquisition switch 3a is in an off-state, the process returns to step S312 and the process stands by until the acquisition switch 3a is turned on.

In step S313, the control unit 5 causes the X-ray generation unit 1 to irradiate the subject 20 with the X ray 1a during the irradiation time Tx, after reading the unnecessary charges occurring due to a dark current. The X-ray image imaging unit 41 performs accumulation of charges during the accumulation time Twx to image the X-ray image. When the X-ray irradiation from the X-ray generation unit 1 ends and the accumulation time Txw elapses, the X-ray image imaging unit 41 images the X-ray image in step S314 and stores the imaged X-ray image in the storage unit 46 in step S315.

Thereafter, in step S316, the present dark image imaging unit 42 images the present dark image, after reading the unnecessary charges occurring due to a dark current. Here, the accumulation time at the time of the imaging of the present dark image is Twt. Next, in step S317, the present dark image imaging unit 42 calculates the average pixel value of the predetermined region of the imaged present dark image and stores the average value in the storage unit 46.

In step S318, the offset correction unit 46 performs the offset correction on the X-ray image based on the X-ray image, the average value of the present dark image, the first preliminary dark image and the average value of the second preliminary dark image, and then process ends.

Hereinafter, the offset correction in step S318 will be described. On the assumption that Vx is the X-ray image imaged in step S314, Vdn is the first preliminary dark image imaged in step S303, and Vo1 is an image corrected based on those images, the corrected image Vo1 can be obtained by subtracting the first preliminary dark image Vdn from the X-ray image Vx in all pixels. This process is expressed by Equation 7 below:

Vo1=Vx−Vdn Equation 7

A variation in a dark current caused due to a difference between the accumulation times can be canceled in the corrected image Vo1 expressed by Equation 7, since the accumulation time at the time of the imaging of the X-ray image Vx is the same as the accumulation time at the time of the imaging of the first preliminary dark image Vdn.

On the assumption that AvrVd is the average pixel value of the present dark image calculated in step S317 and AvrVdtn is the average pixel value of the second preliminary dark image imaged in step S306, corrected image Vo2 can be obtained by subtracting the average pixel value AvrVdtn of the second preliminary dark image from the average pixel value AvrVd of the present dark image. This process is expressed by Equation 8 below:

Vo2=AvrVd−AvrVdtn Equation 8

The corrected image Vo2 expressed by Equation 8 is an offset component occurring due to temperature variation between a temperature at the time of the imaging of the present dark image and a temperature at the time of the imaging of the second preliminary dark image.

Accordingly, an image Vo3 obtained after the offset correction based on the corrected image Vo1 and the corrected image Vo2 is expressed by Equation 9 below:

Vo3=Vo1−Vo2 Equation 9

Through the above-described series of the offset corrections, the charge of the dark current occurring due to the difference between the accumulation times and the temperature variation can be corrected based on Equations 7 to 9.

The sensor 4 transmits the image obtained after the offset correction to the control unit 5 via the communication circuit (not shown). The control unit 5 performs image processing or the like on the image subjected to the offset correction and displays the processed image on the display unit 6.

In this embodiment, the case has been described in which the accumulation time Tw1 at the time of the imaging of the first preliminary dark image is set to 200 ms, 400 ms, and 1000 ms, but the invention is not limited thereto. Any value may be set, for example, the accumulation time Tw1 at the time of the imaging of the first preliminary dark image of the first time may be set to 300 ms, the accumulation time Tw2 at the time of the imaging of the first preliminary dark image of the second time may be set to 800 ms, and the accumulation time Tw3 at the time of the imaging of the first preliminary dark image of the third time may be set to 3000 ms. The number of times the first preliminary dark image and the second preliminary dark image are imaged has been described as three times, but the invention is not limited thereto. The accumulation time Twt at the time of the imaging of the second preliminary dark image has been described as 1 ms, but the invention is not limited thereto.

In this embodiment, the constituent units 41 to 43, and 45 other than the preliminary dark image storage unit 44 are realized by executing the program stored in the memory (not shown). However, for example, the constituent units may be realized by hardware.

In this embodiment, the reading time Tr2 at the time of the imaging of second preliminary dark image is set to be shorter than the reading time Tr1 at the time of the imaging of first preliminary dark image, but the invention is not limited thereto. For example, the reading time Tr2 at the time of the imaging of second preliminary dark image may be set to be same as the reading time Tr1 at the time of the imaging of first preliminary dark image. The reading time of the second dark image can be shortened, for example, by simultaneously reading two lines of the photoelectric conversion elements arrayed in a two-dimensional form.

In this embodiment, the average pixel value of the predetermined region of the second preliminary dark image and the average pixel value of the predetermined region of the present dark image have been calculated, but the invention is not limited thereto. For example, the entire region may be set as the predetermined region. For example, the user may set any region as the predetermined region.

Further, in this embodiment, when the imaging conditions (the tube voltage, the tube current, and the irradiation time) are input in response to a user's operation of the operation input unit 2, the accumulation time at the time of the imaging of the X-ray image has been determined based on the irradiation time in the imaging conditions set in the operation input unit 2 via the control unit 5. However, for example, when the irradiation switch 2a is turned off during the irradiation with the X ray, the control unit 5 detects that the irradiation switch 2a is turned off and immediately stops the irradiation with the X ray from the X-ray generation unit 1. In contrast, for example, the X-ray image imaging unit 41 may image the X-ray image after waiting for only an accumulation time determined in advance. Alternatively, when the irradiation switch 2a is turned off earlier, the X-ray image imaging unit 41 may select the accumulation time which is equal to or greater than the X-ray irradiation time and is the closest to the X-ray irradiation time from among the accumulation times at the time of the reading of the first preliminary dark image, stored in the preliminary dark image storage unit 44. The same process can be applied even in a case in which the X-ray image imaging system is configured to automatically stop the irradiation with the X ray when an amount of X-ray is a predetermined amount.

In this embodiment, the sensor 4 includes the X-ray image imaging unit 41, the present dark image imaging unit 42, the preliminary dark image acquisition unit 43, the preliminary dark image storage unit 44, the offset correction unit 45, and the storage unit 46, but the invention is not limited thereto and the control unit 5 may include a part or all of those constituent units. For example, the control unit 5 may include the X-ray image imaging unit 41, the present dark image imaging unit 42, the preliminary dark image acquisition unit 43, the preliminary dark image storage unit 44, the offset correction unit 45, and the storage unit 46. FIG. 1B illustrates the configuration of the X-ray image imaging system 10′ including a sensor 4′ and a control unit 5′ in this case. Note that, the constituent units and processes thereof in this case are similar to the constituent units and the processes thereof in the X-ray image imaging system 10 according to the first embodiment illustrated in FIG. 1A, and hence the reference numerals in FIG. 1A are also used and description is omitted. In this case, the memory (not shown) for storing the program may also be included in the control unit 5′.

In the first embodiment, the offset correction can reliably be performed and a time from the irradiation of the X ray to image display can be shortened.

Next, a second embodiment of the invention will be described. Since the configuration of an X-ray image imaging system according to the second embodiment is similar to the configuration of the X-ray image imaging system 10 illustrated in FIG. 1A according to the first embodiment, the reference numerals in FIG. 1A are also used in the following description. However, a preliminary dark image storage unit 44 according to the second embodiment stores an image obtained by averaging a plurality of preliminary dark images.

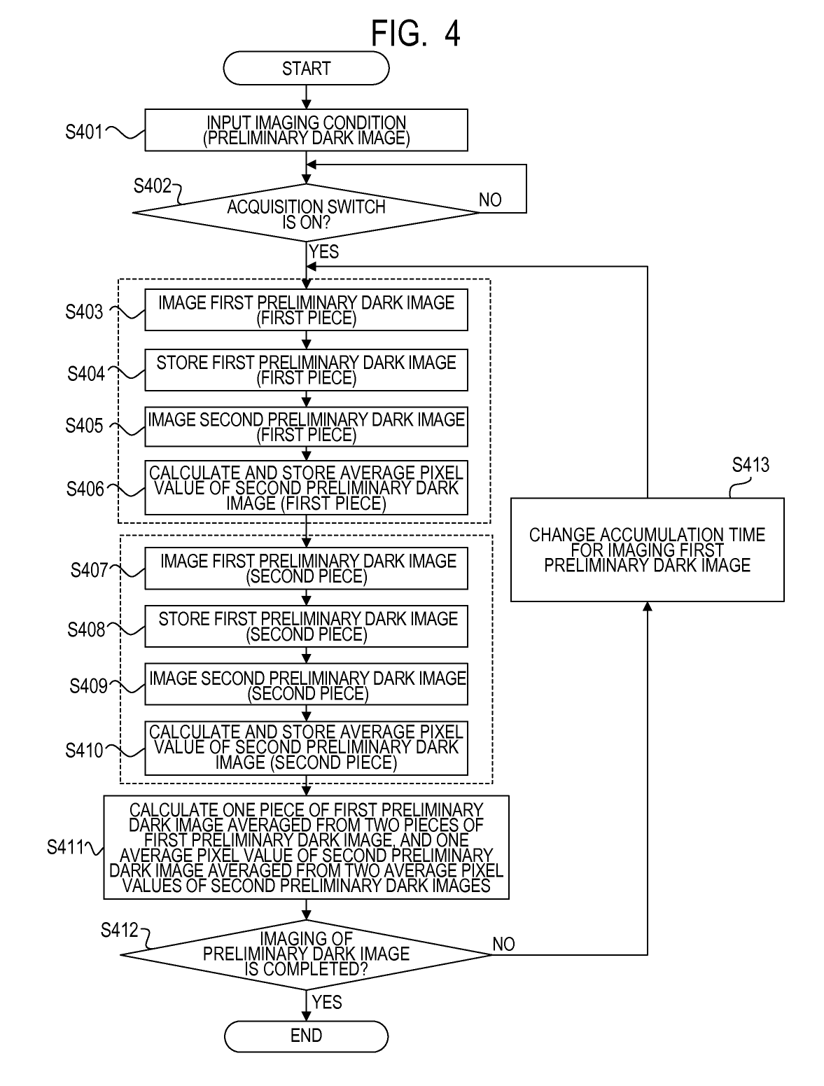

Hereinafter, a process of an X-ray image imaging system 10 according to the second embodiment will be described with reference to FIG. 4. FIG. 4 is a flowchart illustrating a process of imaging two first preliminary dark images and two second preliminary dark images according to the second embodiment of the invention.

In step S401, imaging conditions are input into the preliminary dark image acquisition unit 43 in response to a user's operation of the preliminary dark image operation unit 3 and stored in the preliminary dark image storage unit 44. Here, accumulation times (200 ms and 400 ms) of the first dark image and an accumulation time (1 ms) of the second dark image are input as the imaging conditions.

In step S402, the preliminary dark image acquisition unit 43 determines whether the user operates the acquisition switch 3a and the acquisition switch 3a is turned on. When the acquisition switch 3a is turned on, the process proceeds to step S403. Conversely, when the acquisition switch 3a is in an off-state, the process returns to step S402 and the process stands by until the acquisition switch 3a is turned on.

In step S403, the preliminary dark image acquisition unit 43 images a first piece of first preliminary dark image in the accumulation time of 200 ms input in step S401, after reading the unnecessary charges occurring due to a dark current. In step S404, the preliminary dark image acquisition unit 43 stores the first piece of imaged first preliminary dark image in the preliminary dark image storage unit 44.

In step S405, the preliminary dark image acquisition unit 43 images a first piece of second preliminary dark image in the accumulation time of 1 ms input in step S401, after reading the unnecessary charge occurring due to a dark current. In step S406, the preliminary dark image acquisition unit 43 calculates an average pixel value of the predetermined region of the first piece of second preliminary dark image, and then stores the average pixel value in the preliminary dark image storage unit 44.

In step S407, the preliminary dark image acquisition unit 43 images a second piece of first preliminary dark image in the accumulation time of 200 ms, which is the same as the accumulation time in step S403, after reading the unnecessary charges occurring due to a dark current. In step S408, the preliminary dark image acquisition unit 43 stores the second piece of imaged first preliminary dark image in the preliminary dark image storage unit 44.

In step S409, the preliminary dark image acquisition unit 43 images a second piece of preliminary second dark image in the accumulation time of 1 ms, which is the same as the accumulation time in step S405, after reading the unnecessary charges occurring due to a dark current. In step S410, the preliminary dark image acquisition unit 43 calculates an average pixel value of the predetermined region of the second piece of second preliminary dark image, and then stores the average pixel value in the preliminary dark image storage unit 44.

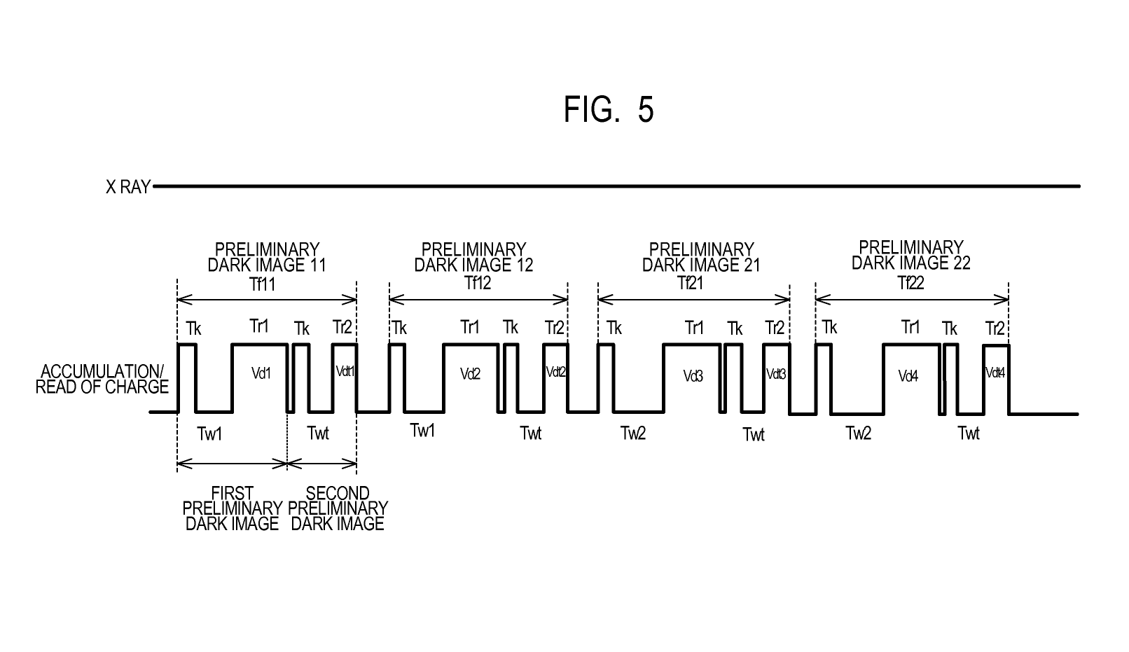

FIG. 5 is a timing chart illustrating a process of imaging the first preliminary dark image and the second preliminary dark image according to the second embodiment of the invention. In the example of the imaging process illustrated in FIG. 5, the accumulation time Tw1 of the first preliminary dark image is set to 200 ms, the accumulation time Twt of the second preliminary dark image is set to 1 ms, and the imaging of first preliminary dark image and the second preliminary dark image is repeated twice. Next, the accumulation time Tw2 of the first preliminary dark image is set to 400 ms (the accumulation time Twt of the second preliminary dark image retains in 1 ms) and the imaging of first preliminary dark image and the second preliminary dark image is repeated twice. Before the accumulation is performed to image the first preliminary dark image and the second preliminary dark image, a process of reading the unnecessary charges occurring due a dark current is performed.

In step S411, the preliminary dark image acquisition unit 43 averages the first preliminary dark images imaged by setting the accumulation time Tw1 to 200 ms and then stores an averaged first preliminary dark image obtained through averaging in the preliminary dark image storage unit 44. In view of this, the preliminary dark image acquisition unit 43 functions as a first calculation unit for calculating a piece of the averaged first preliminary dark image. Further, the preliminary dark image acquisition unit 43 stores the average pixel values of the two second preliminary dark images imaged by setting the accumulation time Tw2 to 1 ms in step S406 and step S410, as described above. The preliminary dark image acquisition unit 43 calculates an average of the average pixel values of the second preliminary dark images obtained through averaging the two average pixel values and stores the averaged average pixel value in the preliminary dark image storage unit 44. In view of this, the preliminary dark image acquisition unit 43 functions as a second calculation unit calculating an averaged average pixel value of second preliminary dark image.

In step S412, the preliminary dark image acquisition unit 43 determines whether the imaging of the first and second preliminary dark images is completed with respect to all of the accumulation times stored in the preliminary dark image storage unit 44 in step S401. When the imaging of the preliminary dark images is not completed with respect to all of the accumulation times, the accumulation time at the time of the imaging of the first preliminary dark image in step S413 is changed and the process returns to step S403. Conversely, when the imaging of the first preliminary dark images is completed with respect to all of the accumulation times, the preliminary dark image acquisition unit 43 ends the process of imaging the first and second preliminary dark images.

When the process proceeds from step S412 and step S413, in step S413, the preliminary dark image acquisition unit 43 changes the accumulation time to the accumulation time in which the imaging is not yet performed among the accumulation times stored in the storage unit 47 in step S401 and imaging the first preliminary dark image in step S403. That is, in this embodiment, by imaging the first preliminary dark image twice in the order of the accumulation times, 200 ms and 400 ms, the process of imaging the first preliminary dark image and the second preliminary dark image ends.

Thereafter, the process of imaging the X-ray image, the process of imaging the present dark images, and the subsequent offset correction process based on the first preliminary dark image and the second preliminary dark image are performed. The processes are similar to the processes described with reference to FIG. 3B. In the offset correction process corresponding to step S318 of FIG. 3B in the second embodiment, however, the averaged first preliminary dark images and the average of the average pixel values of the second preliminary dark images are used. Thus, the offset correction in which the influence of the variation in the dark current contained in the first preliminary dark image and the second preliminary dark image is suppressed can be performed.

In the second embodiment, the case in which two dark images are averaged has been described, but the invention is not limited thereto. Four dark images may be averaged.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

In the above-described embodiments, the offset correction can reliably be performed and the time from the irradiation of the X ray to the image display can be shortened. Accordingly, an effective use of the invention is expected in the medical field in which it is necessary to perform imaging the X-ray image with high quality more rapidly.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Applications No. 2012-133905, filed Jun. 13, 2012, and No. 2013-117392, filed Jun. 3, 2013, which are hereby incorporated by reference herein in their entirety.

What is claimed is:

1. A radiation imaging apparatus comprising:
a first preliminary dark image imaging unit configured to image a first preliminary dark image in each accumulation time, in advance, by accumulating a charge in at least one or more different accumulation times in a detection unit configured to detect a radial ray and accumulate a charge, under a condition that the radial ray is not irradiated by an irradiation unit;
an irradiation control unit configured to control the irradiation unit to irradiate the radial ray to image a radiation image;
a selection unit configured to select an accumulation time corresponding to an irradiation time of the radial ray by the irradiation unit from among the accumulation times;
a setting unit configured to set an accumulation time of a charge in the detection unit when the radiation image is imaged to the accumulation time selected by the selection unit;
a radiation image imaging unit configured to image the radiation image by accumulating the charge in the detection unit in the accumulation time set by the setting unit; and
an offset correction unit configured to perform offset correction on the radiation image based on the first preliminary dark image.

2. The radiation imaging apparatus according to claim 1, further comprising:
a second preliminary dark image imaging unit configured to image a second preliminary dark image by accumulating a charge in a predetermined accumulation time in the detection unit in association with the process of imaging the first preliminary dark image by the first preliminary dark image imaging unit, under the condition that the radial ray is not irradiated by the irradiation unit; and
a present dark image imaging unit configured to image a present dark image by accumulating a charge in the detection unit at the predetermined accumulation time in the detection unit in association with the process of imaging the radiation image by the radiation image imaging unit, under the condition that the radial ray is not irradiated by the irradiation unit,
wherein the offset correction unit performs the offset correction on the radiation image based on also the second preliminary dark image and the present dark image.

3. The radiation imaging apparatus according to claim 2, wherein the accumulation time of the charge in the detection unit when the present dark image is imaged is shorter than the accumulation time of the charge in the detection unit when the radiation image is imaged.

4. The radiation imaging apparatus according to claim 2, wherein the accumulation time of the charge in the detection unit when the second preliminary dark image is imaged is shorter than the accumulation time of the charge in the detection unit when the first preliminary dark image is imaged.

5. The radiation imaging apparatus according to claim 2, wherein a reading time of the charge in the detection unit when the second preliminary dark image is imaged is shorter than a reading time of the charge in the detection unit when the first preliminary dark image is imaged.

6. The radiation imaging apparatus according to claim 1, wherein the offset correction unit performs the offset correction on the radiation image by subtracting the first preliminary dark image from the radiation image.

7. The radiation imaging apparatus according to claim 2, wherein the offset correction unit performs the offset correction on the radiation image by subtracting a value obtained through subtraction of an average pixel value of the second preliminary dark image from an average pixel value of the present dark image, from a value obtained through subtraction of the first preliminary dark image from the radiation image.

8. The radiation imaging apparatus according to claim 7, wherein the average pixel value of the present dark image is an average pixel value in a predetermined partial area of the present dark image.

9. The radiation imaging apparatus according to claim 7, wherein the average pixel value of the second preliminary dark image is an average pixel value in a predetermined partial area of the second preliminary dark image.

10. The radiation imaging apparatus according to claim 1, further comprising:
a first calculation unit configured to calculate an image by averaging a plurality of first preliminary dark images imaged by the first preliminary dark image imaging unit,
wherein the offset correction unit performs the offset correction on the radiation image based on the image calculated by the first calculation unit.

11. The radiation imaging apparatus according to claim 2, further comprising:
a second calculation unit configured to calculate an image by averaging a plurality of second preliminary dark images imaged by the second preliminary dark image imaging unit,
wherein the offset correction unit performs the offset correction on the radiation image based on the image calculated by the second calculation unit.

12. A radiation imaging method executed by a radiation imaging apparatus, the method comprising:
imaging a first preliminary dark image in each accumulation time, in advance, by accumulating a charge in at least one or more different accumulation times in a detection unit configured to detect a radial ray and accumulate a charge, under a condition that the radial ray is not irradiated by an irradiation unit;
controlling the irradiation unit to irradiate the radial ray to image a radiation image;
selecting an accumulation time corresponding to an irradiation time of the radial ray by the irradiation unit from among the accumulation times;
setting an accumulation time of a charge in the detection unit when the radiation image is imaged to the accumulation time selected by the selecting of the accumulation time;
imaging the radiation image by accumulating the charge in detection unit in the accumulation time set by the setting of the accumulation time; and
performing offset correction on the radiation image based on the first preliminary dark image.

13. A program causing a computer to perform:
imaging a first preliminary dark image in each accumulation time in advance by accumulating a charge in at least one or more different accumulation times in a detection unit configured to detect a radial ray and accumulate a charge, under a condition that the radial ray is not irradiated by an irradiation unit;
controlling the irradiation unit to irradiate the radial ray to image a radiation image;
selecting an accumulation time corresponding to an irradiation time of the radial ray by the irradiation unit from among the accumulation times;
setting an accumulation time of a charge in the detection unit when the radiation image is imaged to the accumulation time selected by the selecting of the accumulation time;
imaging the radiation image by accumulating the charge in the detection unit in the accumulation time set by the setting of the accumulation time; and
performing offset correction on the radiation image based on the first preliminary dark image.

14. The radiation imaging apparatus according to claim 1, wherein the selection unit configured to select the accumulation time used to image the radiation image selects an accumulation time which is equal to or greater than the irradiation time of the radial ray and is the closest to the irradiation time of the radial ray from among the one or more different accumulation times when the first preliminary dark image is imaged.

15. A radiation imaging system comprising:
a first preliminary dark image imaging unit configured to image a first preliminary dark image in each accumulation time, in advance, by accumulating a charge in at least one or more different accumulation times in a sensor configured to detect a radial ray and accumulate a charge, under a condition that the radial ray is not irradiated by an irradiation unit;
an irradiation control unit configured to control the irradiation unit to irradiate the radial ray to image a radiation image;
a selection unit configured to select an accumulation time corresponding to an irradiation time of the radial ray by the irradiation unit from among the accumulation times;
a setting unit configured to set an accumulation time of a charge in the sensor when the radiation image is imaged to the accumulation time selected by the selection unit;
a radiation image imaging unit configured to image the radiation image by accumulating the charge in the sensor in the accumulation time set by the setting unit; and
an offset correction unit configured to perform offset correction on the radiation image based on the first preliminary dark image.

16. The radiation imaging system according to claim 15, further comprising:
a second preliminary dark image imaging unit configured to image a second preliminary dark image by accumulating a charge in a predetermined accumulation time in the sensor in association with the process of imaging the first preliminary dark image by the first preliminary dark image imaging unit, under the condition that the radial ray is not irradiated by the irradiation unit; and
a present dark image imaging unit configured to image a present dark image by accumulating a charge in the predetermined accumulation time in the sensor in association with the process of imaging the radiation image by the radiation image imaging unit, under the condition that the radial ray is not irradiated by the irradiation unit,
wherein the offset correction unit performs the offset correction on the radiation image based on also the second preliminary dark image and the present dark image.

17. The radiation imaging system according to claim 15, further comprising:
a control unit,
wherein the control unit includes the offset correction unit.
